SEQUENCE LISTING (2 sequences)

SEQ ID NO: 1 - protein, 793 aa, synthetic construct
MGAIGGLVKINISFIPLFVISASIFIGACNSSKLESGVDSNNISPVMLFDFENDQVPSNIHFLNARASIETYTGINGEPSKGLKLAMQSKQHSYTGLAIVPEQPWDWSEFTSASLYFDIVSVGDHSTQFYLDVTDQNGAVFTRSIDIPVGKMQSYYAKLSGHDLEVPDSGDVNDLNLASGLRSNPPTWTSDDRQFVWMWGVKNLDLSGIAKISLSVQSAMHDKTVIIDNIRIQPNPPQDENFLVGLVDEFGQNAKVDYKGKIHSLEELHAARDVELAELDGKPMPSRSKFGGWLAGPKLKATGYFRTEKINGKWMLVDPEGYPYFATGLDIIRLSNSSTMTGYDYDQATVAQRSADDVTPEDSKGLMAVSEKSFATRHLASPTRAAMFNWLPDYDHPLANHYNYRRSAHSGPLKRGEAYSFYSANLERKYGETYPGSYLDKWREVTVDRMLNWGFTSLGNWTDPAYYDNNRIPFFANGWVIGDFKTVSSGADFWGAMPDVFDPEFKVRAMETARVVSEEIKNSPWCVGVFIDNEKSFGRPDSDKAQYGIPIHTLGRPSEGVPTRQAFSKLLKAKYKTIAALNNAWGLKLSSWAEFDLGVDVKALPVTDTLRADYSMLLSAYADQYFKVVHGAVEHYMPNHLYLGARFPDWGMPMEVVKAAAKYADVVSYNSYKEGLPKQKWAFLAELDKPSIIGEFHIGAMDHGSYHPGLIHAASQADRGEMYKDYMQSVIDNPYFVGAHWFQYMDSPLTGRAYDGENYNVGFVDVTDTPYQEMVDAAKEVNAKIYTERLGSK

SEQ ID NO: 2 - DNA, 2256 bp, synthetic construct
  source 1..2256: mol_type = other DNA
ggatccatgttattcgattttgaaaacgatcaagtcccttcaaatattcattttttaaatgcgcgtgcaagtatagaaacctataccggtataaatggcgagccgagtaaagggttaaagttggcgatgcagtccaagcagcacagttatactggccttgccattgtgccagagcagccttgggattggagcgagtttacctctgctagcttgtatttcgatatagtcagtgttggcgatcattccacacaattttatttagatgttaccgaccaaaatggcgccgtgtttacccgcagtattgatattccagtgggtaaaatgcaatcgtactacgccaagttaagcggtcacgatttagaagtgcccgatagtggagacgttaacgatttaaacctcgcctctggcttgcgttctaacccgcctacatggacatctgacgataggcagtttgtttggatgtggggagtgaaaaatttagatttgtcgggcattgctaaaatatcgctaagtgtgcaaagcgcaatgcacgataaaacagttattatcgataatattcgtattcaacccaacccgccgcaagatgaaaacttccttgtcggtttggtagacgagtttggccaaaacgccaaagttgattacaagggtaaaatccatagtttagaagaattgcatgcagcgcgcgatgtggaactggccgagcttgatggcaagccaatgcctagtcgctctaagtttggcggttggttggccggccccaagctaaaagctacagggtactttcgcacagaaaaaattaacggtaaatggatgctagtagacccagaagggtacccttactttgctacgggtttagacattattcgcctatctaattcatctaccatgactggttacgattacgatcaagctactgttgctcagcgctctgccgacgatgtaacacctgaagactcaaaaggtttaatggcagtgagcgaaaaatcatttgctacgcgccacctagcatcgccaacacgagcggcaatgtttaactggttgccagattacgatcaccctctcgcaaatcattataactaccgtcgctctgcgcattccggcccactgaaacgcggtgaagcctacagcttctacagtgccaaccttgagcgtaaatacggtgaaacttaccccggttcttacttggataagtggcgcgaagtaacggtagacagaatgctaaactggggctttacctcgctaggcaactggactgacccagcatattacgacaacaatcgcataccgtttttcgcgaatggttgggtaataggggattttaaaaccgtatctagcggtgcggatttttggggcgcaatgccagatgtattcgacccagaatttaaagtgcgcgctatggaaacggcacgcgtggtttcagaagaaattaaaaatagcccttggtgcgtaggggtatttatcgataacgaaaaaagcttcggtcgccccgattccgataaggcgcaatacggtattcccattcataccctcggtcgcccaagcgaaggtgtgcctactaggcaggcgtttagtaagctgcttaaagccaaatacaaaactatagccgcgttaaacaatgcctgggggttaaagcttagttcttgggctgagtttgatttgggcgtagatgtaaaagcgctgccggtaaccgatactctgcgcgcagattactcaatgttactttcggcctatgcggaccaatattttaaggtggtacacggcgcggttgaacattacatgccgaaccacttgtatttaggcgcacgctttcctgattggggaatgccaatggaggtagtgaaagctgccgcaaaatacgccgatgtggttagctataattcctacaaagagggcttgcctaagcagaagtgggcttttttagcagagctagataagccgagtataatcggtgagtttcacataggtgctatggatcacggttcgtatcaccccggtttaattcacgctgcgtcgcaggccgatagaggtgaaatgtacaaagattatatgcaatcggtaattgataacccctacttcgtaggcgcgcactggttccagtatatggattcgccattaacgggcagagcttatgatggtgaaaactacaatgtgggttttgtggatgttaccgacacgccgtaccaagaaatggtggatgcagcaaaagaagtaaatgcgaaaatatacaccgaaaggctaggcagcaaataagaattc

(19) United States
(12) Patent Application Publication
(10) Pub. No.: US 2024/0060062 A1
(43) Pub. Date: Feb. 22, 2024

(54) Agarase Mutant with Improved Thermal Stability and Application Thereof

(71) Applicant: Jiangnan University, Wuxi (CN)

(72) Inventors: Jie LONG, Wuxi (CN); Ziying YE, Wuxi (CN); Zhengyu JIN, Wuxi (CN); Cheng Lu, Wuxi (CN); Xingfei Li, Wuxi (CN); Yaoqi TIAN, Wuxi (CN); Yuxiang BAI, Wuxi (CN); Xing ZHOU, Wuxi (CN); Chao QIU, Wuxi (CN); Long CHEN, Wuxi (CN); Zhengjun XIE, Wuxi (CN)

(21) Appl. No.: 18/489,954

(22) Filed: Oct. 19, 2023

Related U.S. Application Data
(63) Continuation of application No. PCT/CN2022/126972, filed on Oct. 24, 2022.

(30) Foreign Application Priority Data
Apr. 8, 2022 (CN) 2022103671638

(51) Int. Cl. C12N 9/38; C12N 15/63

(52) U.S. Cl. CPC C12N 9/2468; C12N 15/63; C12Y 302/01081

(57) ABSTRACT

Disclosed are an agarase mutant with improved thermal stability and application thereof, belonging to the fields of genetic engineering technology and enzyme engineering. The present disclosure provides an agarase mutant, which is obtained by mutating the amino acid at the 86^{th} site, the 373^{rd} site, the 374^{th} site, the 496^{th} site, the 507^{th} site, or the 747^{th} site of agarase with an amino acid sequence as shown in SEQ ID NO.1. The agarase mutant provided by the present disclosure improves the thermal stability and the hydrolytic activity of the agarase. Compared with the wild type enzyme, the mutant enzyme shows excellent heat resistance and can be industrially used at a relatively high temperature, so that the utilization rate of agar raw materials and the yield of oligosaccharides from agar are improved, and the mutant enzyme has a good industrial application prospect.

### REFERENCE TO SEQUENCE LISTING

The instant application contains a Sequence Listing in XML format as a file named “YGHY-2023-04-SEQ.xml”, created on Aug. 7, 2023, of 6 kB in size, and which is hereby incorporated by reference in its entirety.

### TECHNICAL FIELD

The present disclosure relates to an agarase mutant with improved thermal stability and application thereof, belonging to the fields of genetic engineering technology and enzyme engineering.

### BACKGROUND

Red algae are the most productive of all commercial algae and are abundant in the ocean, and agar is the main component of their cell walls. Agar, as a gel and thickener, is recognized as a safe food additive and can also be applied in the pharmaceutical industry, but with low added value. The main structure of agarose is composed of repetitive β-D-galactose and 3,6-anhydrous-α-L-galactose (AHG) disaccharide units which are linked together. It has been reported that the oligosaccharides extracted from agar have various biological activities, including high antioxidant activity, anti-inflammatory activity, prebiotic effect and bacteriostatic and whitening effects, which greatly improves the added value of agar.

The methods of degrading agar to prepare oligosaccharides from agar (divided into agaro-oligosaccharides (AOS) and neoagaro-oligosaccharides (NAOS)) include physical, chemical and enzymatic hydrolysis methods, each with its own unique advantages and disadvantages. In recent years, the enzymatic hydrolysis method has become a research hotspot due to its characteristics of uniform product, simple process, low energy consumption, no pollution, etc. Therefore, enzymatic hydrolysis for producing the oligosaccharides from agar is considered to be the sustainable and most promising method for the commercial production of the oligosaccharides from agar. But when the system temperature is lower than 40° C. (sol-gel transition temperature), agar forms gel, which greatly reduces the efficiency of enzymatic hydrolysis. However, the optimal temperature for most agarases is within a range of 30-40° C., and thus their thermal stability is poor; when the system temperature is higher than 50° C., the activities of the agarases are greatly damaged; and therefore, there are great limitations in the practical industrial application of the agarases.

In the prior art, there are ways to improve the thermal stability of the agarases by means of genetic engineering.

For example, Su, Xu, Yan, Xie, and Lin et al. increased the T50¹⁰ (temperature with a half-life of 10 min) of agarase AgaA derived from Vibrio sp. ZC-1 by 1.5° C. (disclosed in the paper of SU B-M, XU X-Q, YAN R-X, et al. Mutagenesis on the surface of a beta-agarase from Vibrio sp. ZC-1 increased its thermo-stability [J]. Enzyme and Microbial Technology, 2019, 127: 22-31.);

Jang, Lee and Kim et al. obtained a mutant E99K-T307I with Tm increased by 5.2° C. by performing random mutagenesis on agarase AgaB derived from Zobellia galactanivorans (disclosed in the paper of JANG M-K, LEE S W, LEE D-G, et al. Enhancement of the thermostability of a recombinant beta-agarase, AgaB, from Zobellia galactanivorans by random mutagenesis [J]. Biotechnology Letters, 2010, 32 (7): 943-9.);

Shi, Lu, Ma, Fu, and Yu et al. mutated agarase AgaB derived from Pseudoalteromonas sp. CY24 and obtained a mutant S2, whose Tm is 4.6° C. higher than that of the wild type agarase (disclosed in the paper of CHAO S, XINZHI L, CUIPING M, et al. Enhancing the thermostability of a novel beta-agarase AgaB through directed evolution [J]. Applied Biochemistry and Biotechnology, 2008, 151 (1): 51-9.); and

Zhang et al. modified agarase AgWH50C derived from Agarivorans gilvus WH0801, the obtained mutant K621F had a melting temperature (Tm) 0.88° C. higher than that of the wild type agarase, and the optimal temperature was increased from 30° C. to 38° C. (disclosed in the paper of ZHANG P, ZHANG J, ZHANG L, et al. Structure-based design of agarase AgWH50C derived from Agarivorans gilvus WH0801 to enhance thermostability [J]. Applied Microbiology and Biotechnology, 2019, 103 (3): 1289-98.).

However, the thermal stability of the existing agarases still cannot meet the needs of industrial production. Therefore, the development of high heat-resistant agarases will significantly improve the production and application values of the agarases, and is more conducive to the industrialized production of oligosaccharides from agar, which is one of the key technologies to achieve the high value of marine resources.

### SUMMARY

In the early stage, the inventors conducted researches on different agarase which were derived from different sources and produced different oligosaccharides by hydrolysis. It was found that after being combined with agarose substrate, agarase Aga50D (NCBI accession number: ABD81904, sequence as shown in SEQ ID NO.1) derived from Saccharophagus degradans 2-40 had a complete crystal structure (PDB:4BQ2) and produced single neoagarobiose.

After characterizing the wild type agarase derived from S. degradans 2-40 and a mutant provided by the present disclosure, it was found that the single-point mutant greatly improved the heat resistance of agarase, with a maximum increase in apparent melting temperature (Tm) of 23° C., making it an agarase mutant with the largest increase in thermal stability in existing reports. Compared with the previously reported AgWH50C agarase mutant K621F derived from an Agarivorans gilvus WH0801 strain, the mutant provided by the present disclosure has a better heat resistance improvement effect, and the enzyme activity thereof is also improved to a certain extent, which are conducive to improving the utilization rate of agarose raw materials and increasing the yield of neoagarobiose, thus endowing agarase with greater industrial application value.

The present disclosure provides an agarase mutant with improved thermal stability, and an Escherichia coli engineered strain capable of expressing the agarase mutant.

The present disclosure provides an agarase mutant, which is obtained by mutating the amino acid at the 86^{th} site, the 373^{rd} site, the 374^{th} site, the 496^{th} site, the 507^{th} site, or the 747^{th} site of agarase with an amino acid sequence as shown in SEQ ID NO.1.

In one implementation of the present disclosure, the parental enzyme agarase is derived from S. degradans 2-40.

In one implementation of the present disclosure, the nucleotide sequence encoding the parental enzyme agarase is as shown in SEQ ID NO.2.

In one implementation of the present disclosure, the agarase mutant is obtained by mutating the alanine at the 86^{th} site of the agarase with the amino acid sequence as shown in SEQ ID NO.1 to aspartic acid, designated as A86D.

In one implementation of the present disclosure, the amino acid sequence of the mutant enzyme A86D is as shown in SEQ ID NO.3.

In one implementation of the present disclosure, the nucleotide sequence encoding the mutant enzyme A86D is as shown in SEQ ID NO.4.

In one implementation of the present disclosure, the agarase mutant is obtained by mutating the serine at the 373^{rd} site of the agarase with the amino acid sequence as shown in SEQ ID NO.1 to alanine, designated as S373A.

In one implementation of the present disclosure, the agarase mutant is obtained by mutating the phenylalanine at the 374^{th} site of the agarase with the amino acid sequence as shown in SEQ ID NO.1 to tryptophan, designated as F374W.

In one implementation of the present disclosure, the agarase mutant is obtained by mutating the alanine at the 496^{th} site of the agarase with the amino acid sequence as shown in SEQ ID NO.1 to proline, designated as A496P.

In one implementation of the present disclosure, the agarase mutant is obtained by mutating the valine at the 507^{th} site of the agarase with the amino acid sequence as shown in SEQ ID NO.1 to lysine, designated as V507K.

In one implementation of the present disclosure, the agarase mutant is obtained by mutating the serine at the 747^{th} site of the agarase with the amino acid sequence as shown in SEQ ID NO.1 to glutamine, designated as S747Q.

The present disclosure also provides a gene encoding the above agarase mutant.

The present disclosure provides a recombinant vector carrying the above gene.

In one implementation of the present disclosure, the recombinant vector uses any one of pPlc9k, pHIL-S1, pPlcza, and pET28a as an expression vector.

The present disclosure provides a recombinant cell expressing the above mutant, or containing the above gene, or containing the above recombinant vector.

In one implementation of the present disclosure, the recombinant cell takes a prokaryotic cell or a eukaryotic cell as an expression host.

In one implementation of the present disclosure, the recombinant cell uses any one of E. coli BL(21)DE3, Pichia pastoris GS115, P. pastoris KM71, and P. pastoris KM7 as an expression host.

The present disclosure provides a method for preparing the above agarase mutant, and the method includes the following steps:
(1) designing, according to a determined mutation site, a mutation primer for site-directed mutagenesis, carrying out site-directed mutagenesis with the vector carrying the gene encoding agarase as a template, and constructing the vector containing the gene encoding the mutant;
(2) transforming the vector containing the gene encoding the mutant into a microbial cell; and
(3) selecting positive clones for fermentation culture, collecting cells by centrifugation, and carrying out cell-wall breaking to obtain supernatant, i.e., a crude enzyme solution of the agarase mutant.

The present disclosure also provides a method for improving the thermal stability of agarase, and the method includes:

mutating the alanine at the 86^{th} site of the agarase with the amino acid sequence as shown in SEQ ID NO.1 to aspartic acid; or mutating the serine at the 373^{rd} site of the agarase with the amino acid sequence as shown in SEQ ID NO.1 to alanine; or mutating the phenylalanine at the 374^{th} site of the agarase with the amino acid sequence as shown in SEQ ID NO.1 to tryptophan; or mutating the alanine at the 496^{th} site of the agarase with the amino acid sequence as shown in SEQ ID NO.1 to proline; or mutating the valine at the 507^{th} site of the agarase with the amino acid sequence as shown in SEQ ID NO.1 to lysine; or mutating the serine at the 747^{th} site of the agarase with the amino acid sequence as shown in SEQ ID NO.1 to glutamine.

The present disclosure also provides a method for hydrolyzing agar, which includes adding the above mutant or the above recombinant cell to a reaction system containing the agar to carry out a decomposition reaction.

The present disclosure also provides a method for preparing neoagarobiose, where the neoagarobiose is prepared by reacting with the above mutant or the above recombinant cell.

The present disclosure also provides the application of the above mutant, or the above gene, or the above recombinant vector, or the above recombinant cell in preparation of products containing the hydrolyzed agar or preparation of products containing the neoagarobiose.

The present disclosure also provides applications of the above mutant, or the above gene, or the above recombinant vector, or the above recombinant cell in the fields of industry, medicine, biochemistry, and food.

### Beneficial Effects

(1) The present disclosure provides the agarase mutant with improved thermal stability, belonging to the fields of genetic engineering technology and enzyme engineering. The mutant with significantly improved thermal stability is obtained by using the method provided by the present disclosure, making it an agarase mutant with the largest increase in thermal stability in existing reports. Moreover, the enzyme activity of the mutant provided by the present disclosure is improved while the thermal stability thereof is improved.
(2) The enzyme activity of the mutant A86D provided by the present disclosure is 132.55% relative to wild enzyme, the enzyme activity is slightly increased; and the mutant A86D still retains 89.91%, 63.68% and 42.15% of the original enzyme activity after being subjected to incubation at 50° C. for 15 min, 60 min and 6 h, while the wild enzyme only retain 17.14% and 10.79% of the original enzyme activity after being subjected to incubation at 50° C. for 15 min and 60 min, and the enzyme activity of the wild enzyme is completely lost after 6 h of incubation.
(3) The mutant A86D provided by the present disclosure still retains 72.18% of the original enzyme activity after being subjected to incubation at 60° C. for 180 min and still retains 30.83% of the original enzyme activity after being subjected to incubation for 6 h, while the enzyme activity of the wild enzyme is completely lost after 30 min of incubation at 60° C.
(4) Compared with the wild enzyme, the mutant A86D provided by the present disclosure shows superior heat resistance, and can be industrially produced at a higher temperature, so that the utilization rate of agar raw materials and the yield of the oligosaccharides from agar are improved, and the mutant has a good industrial application prospect.

### BRIEF DESCRIPTION OF FIGURES

FIG. 1: Schematic diagram of plasmid construction.

FIG. 2: Enzyme activity characterization of a wild enzyme Aga50D and its mutants.

FIG. 3: Tm characterization of a wild enzyme Aga50D and its mutants.

FIG. 4: SDS-PAGE images of a wild enzyme Aga50D and its mutants after purification; and in the figure, M represents protein molecular weight standard, 1 represents the purified wild type Aga50D, and 2-15 represent the purified mutants, which are PD1, A86D, V172N, K259P, S286D, S373A, F374W, N400R, A496P, V507K, P677H, S705M, Y706F, and S747Q in sequence.

FIG. 5: Thermal stability characterization of a wild enzyme Aga50D and its mutants at 50° C.

FIG. 6: Thermal stability characterization of a wild enzyme Aga50D and its mutants at 60° C.

FIG. 7: Characterization of optimal reaction temperature for a wild enzyme Aga50D and its mutants.

FIG. 8: Optimal reaction pH characterization of a wild enzyme Aga50D and its mutants.

FIG. 9: Characterization of kinetic parameters of a wild enzyme Aga50D and its mutants.

FIG. 10: Analysis on products of a wild enzyme Aga50D and its mutant at 50° C.

FIG. 11: Analysis on products of a wild enzyme Aga50D and its mutant at 60° C.

### DETAILED DESCRIPTION

The pET28a-Aga50D and its mutants involved in the following examples were synthesized by Suzhou GENEWIZ Biotechnology Co., Ltd. The main reagents involved in the following examples: a BCA concentration determination kit was purchased from Beyotime Biotechnology Co., Ltd., gene synthesis was completed by GENEWIZ Biotechnology Co., Ltd., and other commonly used reagents were domestic analytically pure reagents.

The media involved in the following examples are as follows:
LB liquid medium: 1 g of peptone, 0.5 g of a yeast extract and 1 g of NaCl were weighed. The weighed materials were dissolved in deionized water and diluted to 100 mL, and then sterilized at 121° C. for 20 min in an autoclave.
LB solid medium: 1.8% agar powder was added to the LB liquid medium.
LB liquid-resistant medium: kanamycin was added to the LB liquid medium at a final concentration of 50 μg·mL⁻¹.
LB solid medium: kanamycin was added to the LB solid medium at a final concentration of 50 μg·mL⁻¹.

The purification method of the enzymes involved in the following examples is as follows:
(1) Equilibration: a nickel column was equilibrated with a buffer solution which contained 50 mM of Tris-HCl and 500 mM of NaCl, and had a pH of 7.5;
(2) Sample loading: a pretreated sample was loaded at a flow rate of 1 mL/min;
(3) Washing: mixed proteins were washed with a buffer solution which contained 50 mM of Tris-HCl, 500 mM of NaCl and 20 mM of imidazole, and had a pH of 7.5; and
(4) Elution: a buffer solution which contained 50 mM of Tris-HCl, 500 mM of NaCl and 300 mM of imidazole, and had a pH of 7.5 was used for elution, and a target protein was collected to obtain the purified enzyme.

The detection methods involved in the following examples are described below:

Mutant enzyme activity assay:

The enzyme activity was determined by using a 3,5-dinitrosalicylic acid method (DNS). Agarase catalyzed the hydrolysis of agarose under certain conditions to generate reducing sugar, and 3,5-dinitrosalicylic acid and the reducing sugar were reduced under thermal conditions so as to form a brownish red amino complex. Within a certain range, the color depth was proportional to the amount of the reducing sugar, which could be measured at a wavelength of 520 nm to calculate the enzyme activity. Definition of enzyme activity unit: the amount of enzyme required to catalyze the production of 1 μmol of D-galactose per minute at 30° C. under the condition that pH is 7.0 is defined as one activity unit.

Enzyme activity assay steps:
(1) Preheating: 2 mL of a 1 mg·mL⁻¹ agarose solution (with pH of 7.0) was taken and placed in a colorimetric tube.
(2) Reaction: 0.1 mL of an enzyme solution was added, and evenly mixed by shaking for reacting for 20 min; and 1.5 mL of DNS was added to terminate the reaction, and the product was boiled in a water bath for 5 min, and then immediately cooled.
(3) Measurement: the absorbance was measured at a wavelength of 520 nm, and the enzyme activity was calculated.

Determination of Tm Value

Differential scanning fluoremetry (DSF) was used. The natural protein was in a folded state, with the hydrophobic part hidden inside. As the temperature rose, the protein structure gradually disintegrated, exposing the hydrophobic part. At this time, dyes with affinity for the hydrophobic part bound to the protein, showing an increase in the fluorescence signal intensity of the system; when the temperature reached a certain point, the unfolded protein chains aggregated, and the fluorescent dyes could not bind, and returned to the environment or were subjected to fluorescence quenching at a high temperature, resulting in a decrease in the fluorescence signal intensity; and by tracking and detecting changes in fluorescence signals, the Tm of the protein could be determined. The SYPRO Orange dye was diluted 100 times, and 5 μL of the dye was mixed with 20 μL of protein and placed in a 96-well thin-walled PCR plate. Then, in an ABI StepOnePlus real-time fluorescence quantitative PCR instrument system, the obtained mixture was heated from 25° C. to 99° C. to monitor the fluorescence change.

Steps for Determining Thermal Stability of Enzymes at 50° C. or 60° C.
(1) Preheating: 2 mL of an agarose solution (1 mg·mL⁻¹, pH 7.0) was taken and placed in a colorimetric tube, the colorimetric tube was placed in a water bath at 50° C. or 60° C., and heat preservation was carried out for 30 min, 60 min, 90 min, 120 min, 180 min and 6 h, respectively.
(2) Reaction: 0.1 mL of an enzyme solution was added, and evenly mixed by shaking for reacting for 20 min; and 1.5 mL of DNS was added to terminate the reaction, and the product was boiled in a water bath for 5 min, and then immediately cooled.
(3) Measurement: the absorbance was measured at a wavelength of 520 nm, and the enzyme activity was calculated.

Detection Method for Neoagarobiose (NA2)

An enzymatically hydrolyzed sample and a neoagaro-oligosaccharide standard (purchased from Qingdao BZ Oligo Biotech Co., Ltd., with a purity greater than 98%, diluted to 1 mg·mL⁻¹) were tested by ion chromatography (ICS-5000, Thermo Fisher Scientific, USA) after passing through a 0.22 μm filter membrane. The detection conditions were as follows: chromatographic column: a Dionex CarboPac PA-200 anion exchange column, including an analytical column (4 mm×250 mm) and a guard column (4 mm×50 mm); a mobile phase: a 100 mmol·L⁻¹ NaOH solution and a 150 mmol·L⁻¹ NaAc solution, with a flow rate being 0.5 mL·min⁻¹; an amperometric detector adopted four-potential pulse amperometry for detection; and the column temperature was 30° C., and the injection volume was 2 μL.

The present disclosure will be described in detail below in conjunction with the accompanying drawings and examples.

### EXAMPLE 1

### Construction of Mutants

The specific steps are described below.

(1) Construction of a Recombinant Vector pET28a-Aga50D Containing a Wild Type Agarase Aga50D

According to S. degradans 2-40 published by NCBI, the nucleotide sequence encoding a parental enzyme agarase was as shown in SEQ ID NO.2 and sent to GENEWIZ Biotechnology Co., Ltd. for gene synthesis and plasmid recombination. The vector was pET28a, and the recombinant plasmid was named pET28a-Aga50D.

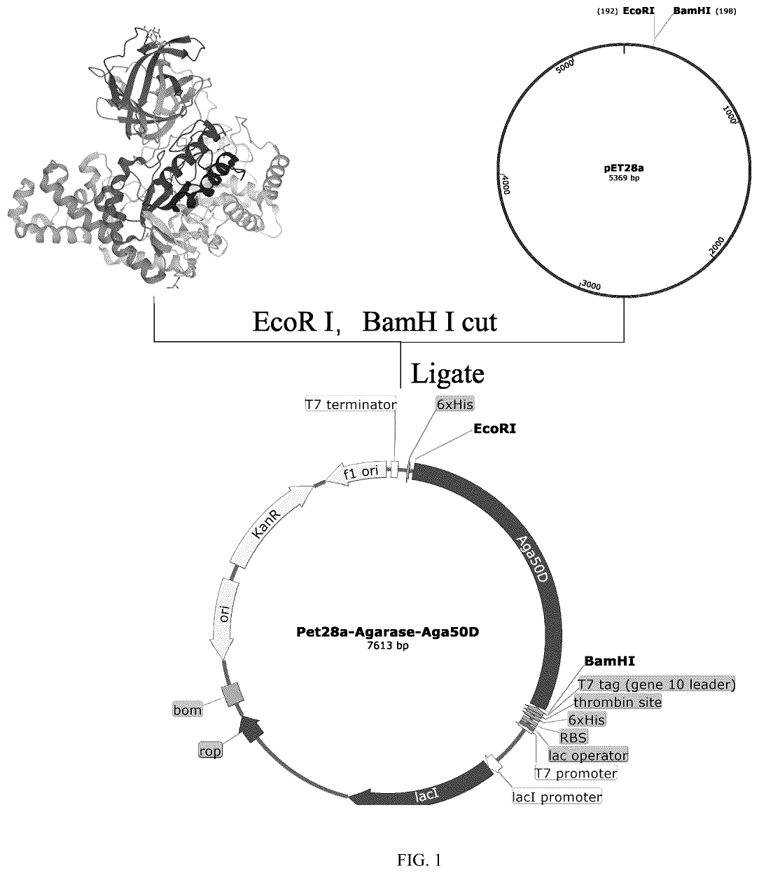

(2) Construction of Recombinant Vectors Containing Mutants (As Shown in FIG. 1)

Site-directed mutagenesis primers were designed to perform site-directed mutagenesis by using the recombinant plasmid pET28a-Aga50D obtained in step (1) as a template so as to obtain recombinant plasmids pET28a-A86D, pET28a-V172N, pET28a-K259P, pET28a-S286D, pET28a-S373A, pET28a-F374W, pET28a-N400R, pET28a-A496P, pET28a-V507K, pET28a-P677H, pET28a-S705M, pET28a-Y706F, and pET28a-S747Q which contain mutants A86D, V172N, K259P, S286D, S373A, F374W, N400R, A496P, V507K, P677H, S705M, Y706F, and S747Q, respectively.

The primer sequences involved are described below.

The site-directed mutagenesis primers for introducing A86D mutation:

A86D-F:
5′-GGTTAAAGTTGGATATGCAGTC-3′;
and
A86D-R:
5′-CTTGGACTGCATATCCAAC-3′.

The site-directed mutagenesis primers for introducing V172N mutation:

V172N-F:
5′-CCCGATAGTGGAGACAACAACG-3′;
and
V172N-R:
5′-GGCGAGGTTTAAATCGTTGTTGTC-3′.

The site-directed mutagenesis primers for introducing K259P mutation:

K259P-F:
5′-CCAAAGTTGATTACCCGGGTAAAATC-3′;
and
K259P-R:
5′-CTAAACTATGGATTTTACCCGGGTAATC-3′.

The site-directed mutagenesis primers for introducing S286D mutation:

S286D-F:
5′-CAAGCCAATGCCTGATCGCTC-3′;
and
S286D-R:
5′-CGCCAAACTTAGAGCGATCAGG-3′.

The site-directed mutagenesis primers for introducing S373A mutation:

S373A-F:
5′-GCAGTGAGCGAAAAAGCTTTTG-3′;
and
S373A-R:
5′-GCGCGTAGCAAAAGCTTTTTC-3′.

The site-directed mutagenesis primers for introducing F374W mutation:

F374W-F:
5′-GAGCGAAAAATCATGGGCTAC-3′;
and
F374W-R:
5′-GCGCGTAGCCCATGATT-3′.

The site-directed mutagenesis primers for introducing N400R mutation:

N400R-F:
5′-CCCTCTCGCACGCCATTATAAC-3′;
and
N400R-R:
5′-CGACGGTAGTTATAATGGCGTGC-3′.

The site-directed mutagenesis primers for introducing A496P mutation:

A496P-F:
5′-GGATTTTTGGGGCCCAATGCC-3′;
and
A496P-R:
5′-CGAATACATCTGGCATTGGGCC-3′.

The site-directed mutagenesis primers for introducing V507K mutation:

V507K-F:
5′-CGACCCAGAATTTAAAAAGCGC-3′;
and
V507K-R:
5′-CGTTTCCATAGCGCGCTTTTTAA-3′.

The site-directed mutagenesis primers for introducing P677H mutation:

P677H-F:
5′-CTACAAAGAGGGCTTGCACAAGC-3′;
and
P677H-R:
5′-GCCCACTTCTGCTTGTGCAAG-3′.

The site-directed mutagenesis primers for introducing S705M mutation:

S705M-F:
5′-GGTGCTATGGATCACGGTATGTATC-3′;
and
S705M-R:
5′-CCGGGGTGATACATACCGTG-3′.

The site-directed mutagenesis primers for introducing Y706F mutation:

Y706F-F:
5′-CTATGGATCACGGTTCGTTTCACC-3′;
and
Y706F-R:
5′-GTGAATTAAACCGGGGTGAAACGAAC-3′.

The site-directed mutagenesis primers for introducing S747Q mutation:

S747Q-F:
5′-CTGGTTCCAGTATATGGATCAACCATTAAC-3′;
and
S747Q-R:
5′-CTCTGCCCGTTAATGGTTGATCC-3′.

The PCR reaction system is as follows:

TABLE 1
Mutation PCR reaction system

| Reagent | Dosage (μL) |
|---|---|
| PrimerSTAR MAX | 25 |
| Upstream primer (10 μM) | 1 |
| Downstream primer (10 μM) | 1 |
| Template (pET28a-Aga50D) | 0.5 |
| ddH₂O | 22.5 |

PCR reaction conditions: pre-denaturation at 95° C. for 3 min, denaturation at 95° C. for 3 min, denaturation at 95° C. for 30 s, annealing at 56° C. for 30 s, and extension at 72° C. for 3 min and 48 s, with a total of 30 cycles.

A target fragment obtained by gel recovery was transformed into E. coli BL21 (DE3), a kanamycin (50 μg/mL) LB plate was coated with a transformant, and the transformant was statically cultured overnight at 37° C. After the bacterial colonies grew out, the single colonies were picked and inoculated into an LB liquid medium containing kanamycin (50 μg/mL), and cultured overnight at 37° C. under the condition of 200 rpm; and the bacterial liquid was sent to GENEWIZ Biotechnology Co., Ltd. for determination. The mutant engineered strains containing the correct mutants were respectively obtained:

E. coli BL21 (DE3)/pET28a-A86D, E. coli BL21 (DE3)/pET28a-V172N, E. coli BL21 (DE3)/pET28a-K259P, E. coli BL21 (DE3)/pET28a-S286D, E. coli BL21 (DE3)/pET28a-S373A, E. coli BL21 (DE3)/pET28a-F374W, E. coli BL21 (DE3)/pET28a-N400R, E. coli BL21 (DE3)/pET28a-A496P, E. coli BL21 (DE3)/-V507K, E. coli BL21 (DE3)/pET28a-P677H, E. coli BL21 (DE3)/pET28a-S705M, E. coli BL21 (DE3)/pET28a-Y706F, and E. coli BL21 (DE3)/pET28a-S747Q; and

according to the above method, the engineered strain E. coli BL21 (DE3)/pET28a-Aga50D containing the original enzyme was prepared.

The combination mutant PD1 was obtained by a combination mutation of 20 sites including A86D, V172N, K259P, V274K, S286D, A351P, A355P, S373A, F374W, A386V, N400R, A496P, V507K, S619E, H635K, P677H, H703R, S705M, Y706F and S747Q based on the pET28a-Aga50D; the primer sequence was the same as above, and the method was the same as above; and the engineered strain E. coli BL21 (DE3)/pET28a-A86D/V172N/K259P/V274K/S286D/A351P/A355P/S373A/F374W/A386V/N400R/A496P/V507K/S619E/H635K/P677H/H703R/S705M/Y706F/S747Q was obtained, and was named E. coli BL21 (DE3)/PD1.

### EXAMPLE 2

### Purification of Enzymes and Determination of Enzymatic Properties

(1) Production of Enzymes by Shake-Flask Fermentation

The genetically engineered strains obtained in Example 1 were respectively streaked on the kanamycin (50 μg/mL) LB plate, and cultured at 37° C. for 12 h; then, single colonies were picked and inoculated into an LB liquid medium containing kanamycin (50 μg/mL) for shake-flask fermentation; and the product was cultured at 37° C. for 12 h under the condition of 200 rpm so as to obtain seed liquid.

1 mL of the seed liquid was absorbed and transferred to an LB liquid medium containing 100 mL of kanamycin (50 μg/mL) for shake-flask fermentation, and then was cultured at 37° C. until OD₆₀₀ reached 0.6-0.8; and IPTG with a final concentration of 0.5 mM was added, the mixture was cooled to 16° C., enzyme was then induced, the product was centrifuged after being cultured for 12 h, and bacterial cells were collected.

(2) Purification of Enzymes

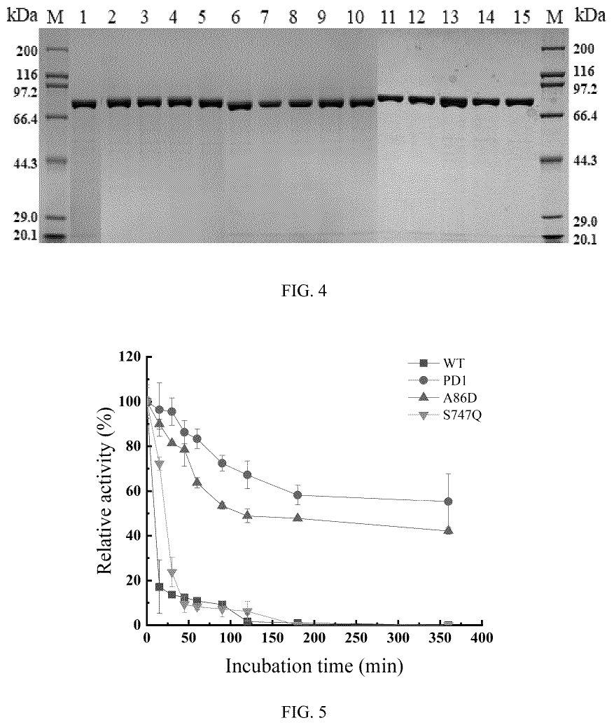

An appropriate amount (2-3 times the volume) of a lysis buffer solution (containing 50 mmol/L Tris HCl and 100 mmol/L NaCl, with a pH of 7.5) was added into the centrifugally collected bacterial cells for resuspending the bacterial cells. The product was subjected to ultrasonication on ice for 15-20 min at a power of 30%; then, the ultrasonication was performed for 2 s, and stopped for 3 s; after the ultrasonication was finished, the product was centrifuged at 4° C. for 10 min under the condition of 8000 rpm to collect supernatant, and the supernatant was filtered through a 0.22 μm filter membrane. Ni²⁺ column affinity chromatography purification was performed to obtain mutant enzymes. After enzyme activity assay and SDS-PAGE electrophoresis detection, the results are as shown in FIG. 4.

Pure enzyme solutions containing wild type agarase and its mutant enzymes A86D, V172N, K259P, S286D, S373A, F374W, N400R, A496P, V507K, P677H, S705M, Y706F, S747Q, and PD1 were prepared, respectively.

(3) Enzyme Activity Assay

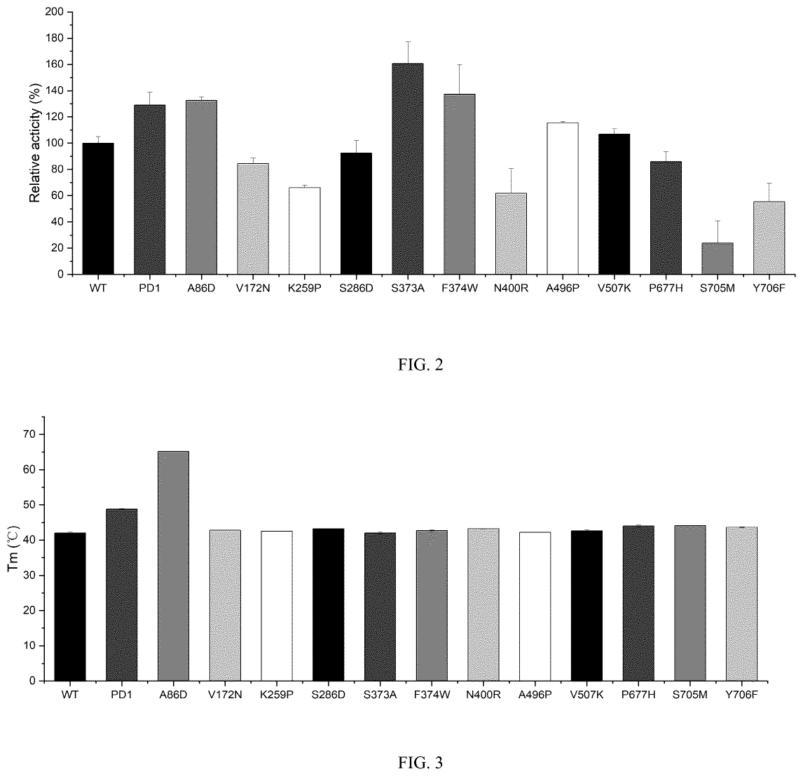

The enzyme activities of the above wild type agarase and its mutant enzymes A86D, V172N, K259P, S286D, S373A, F374W, N400R, A496P, V507K, P677H, S705M, Y706F, S747Q and PD1 were determined. The enzyme activity of the wild type agarase was defined as 100%, and the relative enzyme activities of the mutant enzymes was detected. The results are as shown in Table 2 and FIG. 2.

TABLE 2
Relative enzyme activities of a wild type enzyme and its mutants

| | | | Relative enzyme |
|---|---|---|---|
| Enzyme | Tm | ΔTm | activity (%) |
| WT | 41.974 | — | 100 |
| PD1 | 48.802 | 6.828 | 129.1 ± 9.9 |
| A86D | 65.176 | 23.202 | 132.6 ± 2.5 |
| V172N | 42.768 | 0.793 | 84.6 ± 4.1 |
| K259P | 42.487 | 0.513 | 66.3 ± 1.8 |
| S286D | 43.196 | 1.221 | 92.4 ± 9.8 |
| S373A | 41.987 | 0.013 | 160.7 ± 6.3 |
| F374W | 42.677 | 0.703 | 137.3 ± 10.6 |
| N400R | 43.202 | 1.227 | 61.9 ± 9.7 |
| A496P | 42.210 | 0.236 | 115.5 ± 1.1 |
| V507K | 42.615 | 0.641 | 107.0 ± 3.8 |
| P677H | 44.043 | 2.069 | 86.0 ± 7.4 |
| S705M | 44.137 | 2.163 | 23.8 ± 7.0 |
| Y706F | 43.638 | 1.664 | 55.4 ± 10.4 |
| S747Q | 47.651 | 5.677 | 141.1 ± 4.5 |

The results show that the Tm values of the wild type agarase and its mutant enzymes PD1, A86D, V172N, K259P, S286D, S373A, F374W, N400R, A496P, V507K, P677H, S705M, Y706F, and S747Q are determined by differential scanning fluoremetry, with PD1 (ΔTm=6.828° C.), S747Q (ΔTm=5.677° C.), S705M (ΔTm=2.163° C.), and P677H (ΔTm=2.069° C.) being significant in effect. It is worth noting that the thermal melting temperature (Tm=65.176° C., ΔTm=23.202° C.) of A86D is much higher than that of the wild type agarase (Tm=41.974° C.), so that the heat resistance is greatly improved, as shown in Table 2 and FIG. 3.

Although the enzyme activities of S373A, F374W and A496P have been improved, the increase of their thermal melting temperature is not significant.

Based on the relative enzyme activities, the mutants PD1, A86D, and S747Q with superior overall performance were selected for subsequent experiments.

(4) Enzyme Thermal Stability at 50° C.

The results of an enzyme thermal stability experiment at 50° C. are as shown in FIG. 5.

The results showed that the heat resistance of the mutant A86D was significantly enhanced.

At 50° C., the experimental results showed that the wild enzyme Aga50D only retained 13.65% of the original enzyme activity after being subjected to incubation for 30 min, only retained 10.79% of the original enzyme activity after being subjected to incubation for 60 min, and almost lost all enzyme activity after being subjected to incubation for 180 min.

At 50° C., the mutant S747Q retained 72.17% of the original enzyme activity after being subjected to incubation for 15 min, retained 23.71% of the original enzyme activity after being subjected to incubation for 30 min, and was completely inactivated after being subjected to incubation for 180 min.

At 50° C., the mutant A86D retained 81.39% of the original enzyme activity after being subjected to incubation for 30 min, retained 63.68% of the original enzyme activity after being subjected to incubation for 60 min, and still retained 42.15% of the original enzyme activity after being subjected to incubation for 6 h.

At 50° C., the mutant PD1 retained 95.51% of the original enzyme activity after being subjected to incubation for 30 min, retained 83.37% of the original enzyme activity after being subjected to incubation for 60 min, and still retained 55.28% of the original enzyme activity after being subjected to incubation for 6 h.

Therefore, a heat resistance test at 60° C. was additionally performed to further explore the superior heat resistance performance of PD1 and A86D.

(5) Enzyme Thermal Stability at 60° C.

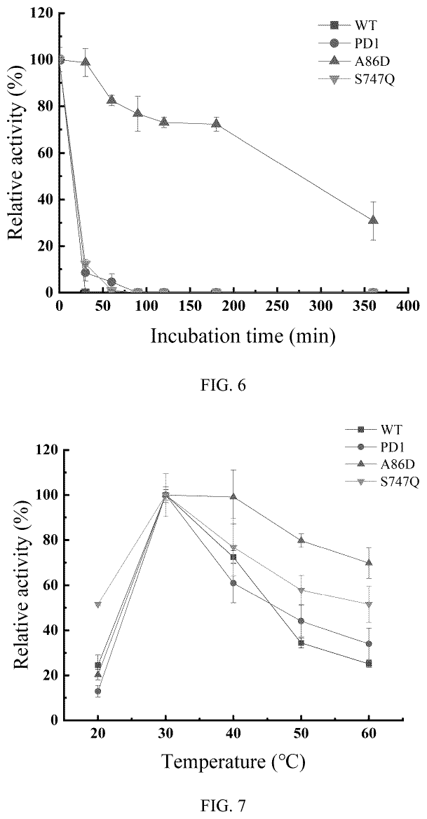

The results of an enzyme thermal stability experiment at 60° C. are as shown in FIG. 6.

The results showed that the heat resistance of the mutant A86D was significantly enhanced.

At 60° C., the experimental results showed that the wild enzyme Aga50D had poor heat resistance, and was completely inactivated after being subjected to incubation at 60° C. for 30 min.

At 60° C., the mutant S747Q retained 12.26% of the original enzyme activity after being subjected to incubation for 30 min, only retained 0.94% of the original enzyme activity after being subjected to incubation for 60 min, and was completely inactivated after being subjected to incubation for 90 min.

At 60° C., the mutant PD1 retained 8.50% of the original enzyme activity after being subjected to incubation for 30 min, only retained 4.50% of the original enzyme activity after being subjected to incubation for 60 min, and was completely inactivated after being subjected to incubation for 90 min.

At 60° C., the mutant A86D retained 98.75% of the original enzyme activity after being subjected to incubation for 30 min, retained 82.46% of the original enzyme activity after being subjected to incubation for 60 min, retained 72.18% of the original enzyme activity after being subjected to incubation for 180 min, and still retained 30.83% of the original enzyme activity after being subjected to incubation for 6 h, thus showing extremely superior heat resistance and having excellent industrial application potential.

### EXAMPLE 3

### Determination of Enzymatic Properties

(1) Optimal Reaction Temperature

In order to further explore the characteristics of mutant enzymes, the enzymatic properties of PD1, A86D and S747Q were further studied. A 0.1% agarose substrate was prepared by using a buffer solution which contained Tris-HCl and had a pH of 7, and an enzyme solution with a final concentration of 0.2 mg·mL⁻¹ was added to react for 20 min at different temperatures so as to measure the enzyme activity. It can be seen from FIG. 7 that the optimal reaction temperature for both a wild enzyme and its mutants is 30° C.

(2) Optimal Reaction pH

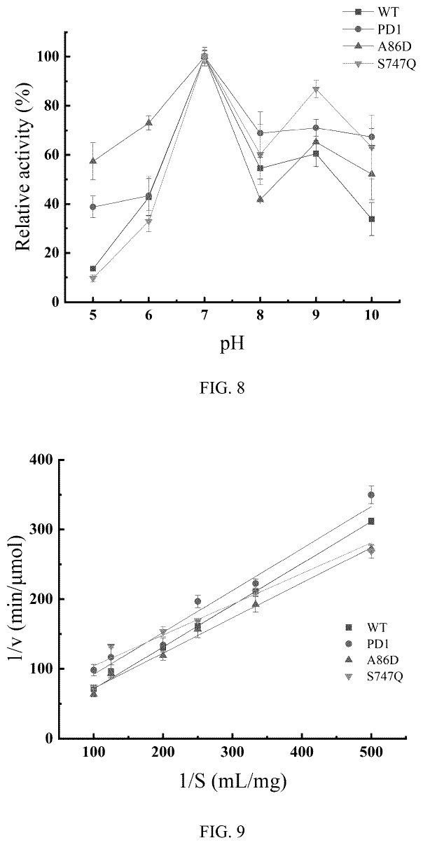

A 1 mg·mL⁻¹ agarose substrate having a pH within a range of 5-6 was prepared by using a buffer solution containing 50 mM of citric acid, a 0.1% agarose substrate having a pH within a range of 7-8 was prepared by using a buffer solution containing 50 mM of Tris-HCl, and a 0.1% agarose substrate having a pH within a range of 9-10 was prepared by using a glycine-NaOH buffer solution. An enzyme solution with a final concentration of 0.2 mg·mL⁻¹ was added to react for 20 min at different temperatures so as to measure the enzyme activity. It can be seen from FIG. 8 that the optimal reaction pH for both a wild enzyme and its mutants is 7, and their properties are similar.

(3) Kinetic Parameters

Agarose substrates with concentrations of 1 mg·mL⁻¹, 2 mg·mL⁻¹, 3 mg·mL⁻¹, 4 mg·mL⁻¹, 5 mg·mL⁻¹, 8 mg·mL⁻¹ and 10 mg·mL⁻¹ were respectively prepared by using a buffer solution which contained Tris-HCl and had a pH of 7. An enzyme solution with a final concentration of 0.2 mg·mL⁻¹ was added to react under the optimal reaction conditions, and then the amount of reducing sugar produced was determined and plotted according to a Lineweaver-Burk method (FIG. 9). The linear fitting equations for the 4 curves in the figure are γ=589.92x+15.37 (WT), γ=621.46x+31.568 (PD1), γ=506.45x+22.215 (A86D), and γ=436.23x+58.247 (S747Q), respectively. Thus, various kinetic parameters of a wild enzyme and its mutants can be deduced (Table 3).

The kinetic parameters of a wild enzyme and its mutants under optimal reaction conditions are listed in Table 3.

TABLE 3
Kinetic parameters of wild type Aga50D and its mutants

| | | Kcat/Km |
|---|---|---|
| | Km (mg/ml) | (mL/mg·s) |
| WT | 38.381 | 0.139 |
| PD1 | 19.686 | 0.154 |
| A86D | 22.794 | 0.162 |
| S747Q | 7.489 | 0.181 |

It can be seen from the data in the table that the Km values of mutants are lower than that of WT, indicating that the binding ability of the mutants to an agarose substrate is significantly increased, which is consistent with the previous conclusion about the relative enzyme activity. The Kcat/Km values of the mutants are greater than that of the WT, indicating that the catalytic ability of the mutants is improved.

### EXAMPLE 4

### Application of Enzymes

After a 1 mg·mL⁻¹ agarose substrate solution was prepared by using ultrapure water, the mutant and wild type pure enzyme solutions (0.2 mg·mL⁻¹ enzyme solution (9.528 U·mg⁻¹)) prepared in step (2) of Example 2 were respectively added to react in a water bath shaker at 50° C., with a rotational speed of 200 rpm and a reaction time of 1-3 h. After the reaction was completed, a boiling water bath was used to terminate the reaction.

Part of the reaction solution was taken out every 1 h, 2 h and 3 h, unreacted polysaccharides in the reaction solution was removed by centrifugation, and supernatant was taken to obtain enzymatic hydrolysate samples: neoagarobiose (NA2). The contents of the neoagarobiose (NA2) in the above-obtained products were detected, respectively.

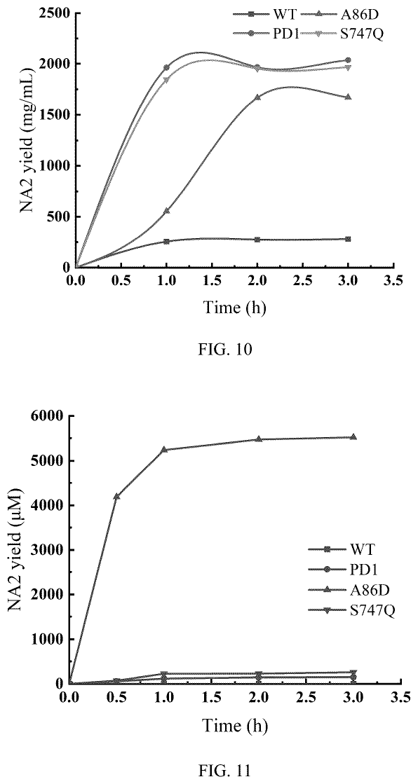

The results show (as shown in FIG. 10) that at 50° C., neoagarobiose produced by hydrolyzing agarose with a wild type enzyme is low in yield, which is presumed to be due to heat instability, and rapid heat inactivation; while mutant enzymes PD1, A86D, and S747Q have certain heat resistance, and can still play a catalytic role under high temperature conditions; and an agarose substrate is still in a solution state at 50° C., which promotes the binding of the substrate to the enzyme, thus further improving the yield of the neoagarobiose.

The hydrolysis reaction lasted for 3 h, and the yield of the product neoagarobiose (NA2) obtained by hydrolyzing agarose with the wild type enzyme was 278.678 mg/mL (859.3 μM);

the yield of the product neoagarobiose (NA2) obtained by hydrolyzing agarose with the mutant enzyme PD1 was 2036.281 mg/mL (6279.3 μM);

the yield of the product neoagarobiose (NA2) obtained by hydrolyzing agarose with the mutant enzyme A86D was 1670.887 mg/mL (5152.6 μM); and

the yield of the product neoagarobiose (NA2) obtained by hydrolyzing agarose with the mutant enzyme S747Q was 1965.719 mg/mL (6061.7 μM).

Under the condition of 50° C., the mutant enzymes show excellent hydrolysis efficiency due to its superior heat resistance, thus greatly improving the yield of neoagarobiose, and having good industrial application value.

### EXAMPLE 5

### Application of Enzymes

After a 1 mg·mL⁻¹ agarose substrate solution was prepared by using ultrapure water, the mutant and wild type pure enzyme solutions (0.2 mg·mL⁻¹ enzyme solution (9.528 U·mg⁻¹)) prepared in step (2) of Example 2 were respectively added to react in a water bath shaker at 60° C., with a rotational speed of 200 rpm and a reaction time of 1-3 h. After the reaction was completed, a boiling water bath was used to terminate the reaction.

Part of the reaction solution was taken out every 1 h, 2 h and 3 h, unreacted polysaccharides in the reaction solution was removed by centrifugation, and supernatant was taken to obtain enzymatic hydrolysate samples: neoagarobiose (NA2). The contents of the neoagarobiose (NA2) in the above-obtained products were detected, respectively.

The results show (as shown in FIG. 11) that under the condition of 60° C., due to the heat instability and rapid inactivation of the wild type enzyme, no product neoagarobiose (NA2) is detected; and the mutants PD1 and S747Q fail to withstand the high temperature of 60° C., resulting in a large loss of enzyme activity. However, A86D can withstand a high temperature of 60° C., and still retains better enzyme activity at 60° C.; and an agarose substrate is still in a solution state at 60° C., which promotes the binding of the substrate to the enzyme, thus further improving the yield of the neoagarobiose.

The hydrolysis reaction lasted for 3 h, and the yield of the product neoagarobiose (NA2) obtained by hydrolyzing agarose with the wild type enzyme was 0 μM;

the yield of the product neoagarobiose (NA2) obtained by hydrolyzing agarose with the mutant enzyme PD1 was 147.6 μM;

the yield of the product neoagarobiose (NA2) obtained by hydrolyzing agarose with the mutant enzyme S747Q was 254.9 μM; and

the yield of the product neoagarobiose (NA2) obtained by hydrolyzing agarose with the mutant enzyme A86D was 5513.6 μM.

Under the condition of 60° C., the mutant enzyme A86D shows excellent hydrolysis efficiency due to its superior heat resistance, thus greatly improving the yield of neoagarobiose, and having good industrial application value.

Although the present disclosure has been disclosed as above in exemplary embodiments, it is not intended to limit the present disclosure. Anyone familiar with this technology can make various changes and modifications without departing from the spirit and scope of the present disclosure. Therefore, the protection scope of the present disclosure shall be as defined in the Claims.

What is claimed is:

1. An agarase mutant, wherein the agarase mutant comprises mutation of the amino acid at the 86^{th} site, the 373^{rd} site, the 374^{th} site, the 496^{th} site, the 507^{th} site, or the 747^{th} site of agarase, with an amino acid sequence as set forth in SEQ ID NO.1; the agarase mutant is obtained by mutating the alanine at the 86^{th} site of the agarase with the amino acid sequence as set forth in SEQ ID NO.1 to aspartic acid; or the agarase mutant is obtained by mutating the serine at the 373^{rd} site of the agarase with the amino acid sequence as set forth in SEQ ID NO.1 to alanine; or the agarase mutant is obtained by mutating the phenylalanine at the 374^{th} site of the agarase with the amino acid sequence as set forth in SEQ ID NO.1 to tryptophan; or the agarase mutant is obtained by mutating the alanine at the 496^{th} site of the agarase with the amino acid sequence as set forth in SEQ ID NO.1 to proline; or the agarase mutant is obtained by mutating the valine at the 507^{th} site of the agarase with the amino acid sequence as set forth in SEQ ID NO.1 to lysine; or the agarase mutant is obtained by mutating the serine at the 747^{th} site of the agarase with the amino acid sequence as set forth in SEQ ID NO.1 to glutamine.

2. A recombinant cell expressing the agarase mutant according to claim 1, or containing a gene encoding the agarase mutant, or containing a recombinant vector carrying a gene encoding the agarase mutant.

3. The recombinant cell according to claim 2, wherein the recombinant cell takes a prokaryotic cell or a eukaryotic cell as an expression host.

4. A method for preparing neoagarobiose, wherein the neoagarobiose is prepared by hydrolyzing agarose with the agarase mutant according to claim 1.
